(19) United States
(12) Patent Application Publication
(10) Pub. No.: US 2022/0113233 A1
(43) Pub. Date: Apr. 14, 2022

(54) PARTICLE CAPTURE DEVICE, PARTICLE CAPTURE METHOD, AND MICROSCOPE SYSTEM

(71) Applicant: SONY GROUP CORPORATION, TOKYO (JP)

(72) Inventors: YOSHIAKI KATO, TOKYO (JP); TASUKU YOTORIYAMA, TOKYO (JP)

(21) Appl. No.: 17/424,214

(22) PCT Filed: Dec. 4, 2019
(86) PCT No.: PCT/JP2019/047345

§ 371 Date: Jul. 20, 2021

(30) Foreign Application Priority Data
Jan. 28, 2019 (JP) 2019-011786

(51) Int. Cl. G01N 1/40; B01L 3/00; G02B 21/34

(52) U.S. Cl. CPC G01N 1/4077; B01L 3/502761; B01L 2300/0864; B01L 2200/0652; B01L 2300/027; G02B 21/34

(57) ABSTRACT

To provide a single particle capture technique that can shorten cell capture time without damaging a cell.

The present technology provides a particle capture device including: a particle capture unit having a particle capture region including a plurality of wells that captures particles, and dividing a space into a first space and a second space; a particle supply channel which is connected to the first space and through which a fluid containing the particles is supplied; a first discharge channel which is connected to the first space and through which a fluid is discharged from the first space; and a second discharge channel which is connected to the second space and through which a fluid is discharged from the second space, in which the particles are captured in the wells by simultaneous discharge of fluids from the first discharge channel and the second discharge channel.

### TECHNICAL FIELD

The present technology relates to a particle capture device, a particle capture method, and a microscope system. More specifically, the present technology relates to a particle capture device in which a particle is captured in a well by suction, a particle capture method including a particle capture step of capturing a particle in a well by suction, and a microscope system including the particle capture device.

### BACKGROUND ART

Attention is focused on a single cell analysis technique. In the single cell analysis technique, one cell is captured in each of many microwells arranged on a plane, and the mode of each cell is individually observed to analyze the characteristics of each cell and/or to analyze a reaction of each cell with a reagent using, for example, fluorescence and the like as an index.

Examples of a commercially available device used in the single cell analysis technique include an AS ONE cell picking system (AS ONE Corporation). In an analysis technique using this device, a cell suspension is supplied to a microchamber having many wells each having a size for one cell, and one cell is settled in each of the wells. Then, one cell in each well is collected and/or analyzed individually. The wells are provided on a tip in the microchamber. As the chip, a plurality of types of chips according to a cell size is prepared. For example, a chip in which φ30 μm wells are arranged at a pitch of 80 μm in X and Y directions (about 80,000 wells) and a chip in which φ10 μm wells are arranged at a pitch of 30 μm in X and Y directions (about 300,000 wells) are prepared. The characteristics of each of cells isolated in each well are observed by a means such as fluorescence detection with this device. Then, a cell of interest is extracted from a well by a micromanipulator, is transferred to a 96-well/384-well plate, and then can be subjected to more detailed analysis, such as sequencing.

Furthermore, examples of a technique for capturing one cell in one well include a technique described in Patent Document 1 below. The following Patent Document 1 describes “a micro fluid device that can capture a circulation tumor cell (CTC) contained in a blood sample with a size-selective micro cavity array, the micro fluid device including: an upper member having a sample inlet, a sample outlet, and a microchannel communicating the sample inlet and the sample outlet with each other, and having an opening window for the size-selective micro cavity array at a position corresponding to a part of a microchannel; a micro cavity array support portion including the size-selective micro cavity array having fine through holes for capturing CTC of which hole diameter, hole number, and arrangement are controlled, and a tight seal for supporting the size-selective micro cavity array at a position corresponding to a lower side of the opening window of the upper member; and a lower member having a sucking opening window disposed at a position corresponding to a lower side of the size-selective micro cavity array, and a suction channel communicating the sucking opening window and a suction opening with each other”.

### CITATION LIST

### Patent Document

Patent Document 1: Japanese Patent Application Laid-Open No.

### SUMMARY OF THE INVENTION

### Problems to be Solved by the Invention

In Patent Document 1 described above, a technique for capturing one cell in each well is proposed. In the technique, a hole is formed in a well, and a cell is captured by suction through the hole. With this technique, capture into a well can be performed more efficiently. However, if a suction pressure or a cell (collision) speed at the time of capture is a certain value or more, the cell may enter a suction hole or may be damaged by an impact of the collision disadvantageously, and there is a limit for increasing the suction pressure at a bottom hole of a well.

Furthermore, when a cell is captured in a well, a part of an outlet channel is blocked. Therefore, an overall flow velocity is gradually reduced. Therefore, it takes time to capture cells in a well array at a desired packing density disadvantageously. Moreover, many cells that are not captured in wells and stay between the wells appear due to the reduced flow velocity disadvantageously.

Therefore, an object of the present technology is to provide a single particle capture technique that can shorten cell capture time without damaging a cell.

### Solution to Problems

The inventors of the present application have found that the above problems can be solved by devising a flow of a fluid, and have completed the present technology.

That is, the present technology provides a particle capture device including:

a particle capture unit having a particle capture region including a plurality of wells that captures particles, and dividing a space into a first space and a second space;

a particle supply channel which is connected to the first space and through which a fluid containing the particles is supplied;

a first discharge channel which is connected to the first space and through which a fluid is discharged from the first space; and

a second discharge channel which is connected to the second space and through which a fluid is discharged from the second space, in which

the particles are captured in the wells by simultaneous discharge of fluids from the first discharge channel and the second discharge channel.

In the particle capture device according to the present technology, the first discharge channel can be disposed at a position facing the particle supply channel with the particle capture region interposed therebetween.

During particle capture using the particle capture device according to the present technology, a flow velocity of a fluid flowing through the first discharge channel can be equal to or less than a flow velocity of a fluid flowing through the second discharge channel.

In this case, during particle capture, the flow velocity of a fluid flowing through the first discharge channel : the flow velocity of a fluid flowing through the second discharge channel can be set to 1:1 to 100.

During particle capture using the particle capture device according to the present technology, a suction pressure applied to the first discharge channel and/or a suction pressure applied to the second discharge channel can be changed at a predetermined cycle.

In the particle capture device according to the present technology, the first space can have a larger cross-sectional area toward a downstream side.

In the particle capture device according to the present technology, holes are formed in the wells, and the wells and the second space can be communicated with each other through the holes.

During particle capture using the particle capture device according to the present technology, the first space can be formed above the second space in the direction of gravity.

In the particle capture device according to the present technology, a fluid can be discharged by being sucked through the first discharge channel and the second discharge channel.

Next, the present technology provides

a particle capture method including:

a particle supply step of supplying a fluid containing particles to a first space; and

a particle capture step of capturing particles in a plurality of wells in a particle capture region formed in a particle capture unit dividing a space into the first space and a second space by simultaneously performing discharge of a fluid from the first space and discharge of a fluid from the second space.

In the particle capture method according to the present technology, a discharge velocity of a fluid from the first space can be equal to or less than a discharge velocity of a fluid from the second space.

In the particle capture method according to the present technology, in the particle capture step, the discharge of a fluid from the first space and the discharge of a fluid from the second space can be performed by suction.

The present technology further provides a microscope system including:

a particle capture device including:

a particle capture unit having a particle capture region including a plurality of wells that captures particles, and dividing a space into a first space and a second space;

a particle supply channel which is connected to the first space and through which a fluid containing the particles is supplied;

a first discharge channel which is connected to the first space and through which a fluid is discharged from the first space; and

a second discharge channel which is connected to the second space and through which a fluid is discharged from the second space,

the particles being captured in the wells by simultaneous discharge from the first discharge channel and the second discharge channel; and

an observation unit that observes particles captured in the wells.

The microscope system according to the present technology can further include an analysis unit that analyzes the particles on the basis of information acquired from the observation unit.

During particle capture using the microscope system according to the present technology,

a first discharge control unit that controls discharge via the first discharge channel, and

a second discharge control unit that controls discharge via the second discharge channel

can be further included.

In the present technology, particles are required to be captured one by one, for example. Examples of the particle include, but are not limited to, a biological fine particle such as a cell, a microorganism, a living body-derived solid component, or a liposome, and a synthetic particle such as a latex particle, a gel particle, or an industrial particle. The cell can include an animal cell and a plant cell. Examples of the animal cell include a tumor cell and a blood cell. The microorganism can include bacteria such as Escherichia coli and fungi such as yeast. Examples of the living body-derived solid component include a solid crystal produced in a living body. The synthetic particle may be a particle constituted by, for example, an organic or inorganic polymer material or a metal. The organic polymer material can include polystyrene, styrene-divinylbenzene, polymethyl methacrylate, and the like. The inorganic polymer material can include glass, silica, a magnetic material, and the like. The metal can include gold colloid, aluminum, and the like. Furthermore, in the present technology, the particle may be a combination of a plurality of particles such as two or three particles.

### BRIEF DESCRIPTION OF DRAWINGS

FIG. 1 is a schematic conceptual diagram schematically illustrating a first embodiment of a particle capture device 100 according to the present technology.

FIG. 2 is a schematic conceptual diagram schematically illustrating a second embodiment of the particle capture device 100 according to the present technology.

FIG. 3 is a schematic conceptual diagram schematically illustrating a third embodiment of the particle capture device 100 according to the present technology.

FIG. 4 is a schematic conceptual diagram schematically illustrating a fourth embodiment of the particle capture device 100 according to the present technology.

FIG. 5 is a schematic conceptual diagram schematically illustrating a fifth embodiment of the particle capture device 100 according to the present technology.

FIG. 6 is a schematic conceptual diagram schematically illustrating a sixth embodiment of the particle capture device 100 according to the present technology.

FIG. 7 is a flowchart of a particle capture method according to the present technology.

FIG. 8 is a block diagram of a microscope system 200 according to the present technology.

### MODE FOR CARRYING OUT THE INVENTION

Hereinafter, a preferred mode for carrying out the present technology will be described with reference to the drawings. The embodiments described below exemplify representative embodiments of the present technology, and the scope of the present technology is not narrowly interpreted by the embodiments. Note that the description will be made in the following order.

1. Particle capture device 100

(1) Particle capture unit 103

[Well 108]

[Hole 114]

(2) First space 101

[Particle supply channel 104]

[First discharge channel 105]

[First fluid supply channel 110]

(3) Second space 102

[Second discharge channel 106]

[Second fluid supply channel 111]

2. Particle capture method

(1) Preparation step S1

(2) Particle supply step S2

(3) Particle capture step S3

(4) Particle removal step S4

(5) Particle observation step S5

(6) Particle analysis step S6

(7) Particle treatment step S7

(8) Target particle acquisition step S8

(9) Particle collection step S9

3. Microscope system 200

(1) Observation unit 201

(2) Analysis unit 202

(3) Control unit 203

(4) Storage unit 204

(5) Display unit 205

1

. Particle Capture Device 100

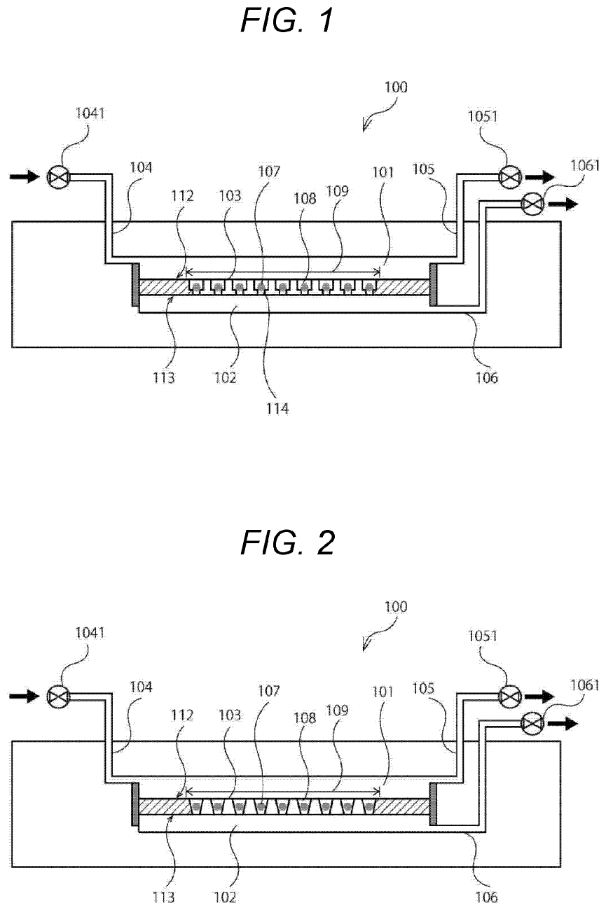

FIG. 1 is a schematic conceptual diagram schematically illustrating a first embodiment of a particle capture device 100 according to the present technology. The particle capture device 100 according to the present technology roughly includes a first space 101, a second space 102, and a particle capture unit 103. To the first space 101, at least a particle supply channel 104 and a first discharge channel 105 are connected. To the second space 102, at least a second discharge channel 106 is connected. Furthermore, the particle capture unit 103 has a particle capture region 109 including a plurality of wells 108 that captures particles 107. Moreover, the particle capture device 100 according to the present technology may include a first fluid supply channel 110, a second fluid supply channel 111, a first discharge control unit 203, a second discharge control unit 203, and the like, if necessary.

In the particle capture device 100 according to the present technology, a fluid containing the particles 107 is supplied to the first space 101 through the particle supply channel 104, and a fluid is discharged from the second space 102 through the second discharge channel 106. As a result, the particles 107 are captured in the wells 108. In the present technology, when the particles 107 are captured in the wells 108, simultaneously with discharge of a fluid from the second discharge channel 106, a fluid is discharged also from the first discharge channel 105 connected to the first space 101.

In a conventional single particle capture technique, only discharge of a fluid from the second discharge channel 106 is performed during particle capture in the wells 108. Therefore, the velocity of a fluid in the first space 101 is lower as a distance from the particle supply channel 104 is larger. Therefore, it may take a long time to make uniform capture possible such that the particles 107 are captured even by the wells 108 away from the particle supply channel 104, or the particles 107 do not reach the wells 108 away from the particle supply channel 104 in some cases.

Meanwhile, in the present technology, when the particles 107 are captured in the wells 108, simultaneously with discharge of a fluid from the second discharge channel 106, a fluid is discharged also from the first discharge channel 105 connected to the first space 101. Therefore, the velocity of a fluid in the first space 101 can be kept constant regardless of a distance from the particle supply channel 104. As a result, the particles 107 can be uniformly guided even to the wells 108 away from the particle supply channel 104, and capture time of the particles 107 can be shortened.

Furthermore, in the conventional single particle capture technique, in order to make uniform capture possible such that the particles 107 are captured even by the wells 108 away from the particle supply channel 104 in a short time, it is necessary to increase a discharge velocity of a fluid from the second discharge channel 106 connected to the second space 102. However, when the discharge velocity of the fluid is increased, the particles 107 to be captured may enter holes 114 in the wells 108 or may be damaged by an impact of collision or the like.

Meanwhile, in the present technology, when the particles 107 are captured in the wells 108, simultaneously with discharge of a fluid from the second discharge channel 106, a fluid is discharged also from the first discharge channel 105 connected to the first space 101. Therefore, without increasing the discharge velocity of a fluid from the second discharge channel 106 connected to the second space 102, the particles 107 can be uniformly guided even to the wells 108 away from the particle supply channel 104. Therefore, it is possible to prevent the particles 107 to be captured from entering the holes 114 in the wells 108 or being damaged.

Hereinafter, each unit will be described in detail.

(1) Particle capture unit 103

The particle capture unit 103 divides a space into the first space 101 and the second space 102. The particle capture unit 103 can be constituted by, for example, a plate-shaped member constituted by a surface 112 on an inlet side of the particles 107 of the wells 108 and a surface 113 facing the surface 112. This makes manufacture of the particle capture unit 103 easier and makes it easier to measure and/or observe the captured particles 107. Furthermore, the ratio of the volume of the particle capture unit 103 to the particle capture device 100 is also reduced, and the entire particle capture device 100 can be downsized.

The thickness of the plate-shaped member can be appropriately set depending on, for example, the depth of the well 108, the depth of the hole 114, and the strength of a material of the plate-shaped member. The thickness of the plate-shaped portion can be, for example, 10 μm to 1000 μm, preferably 15 μm to 500 μm, and more preferably 20 μm to 200 μm.

As a material of the particle capture unit 103 (particularly a portion where the wells 108 are formed), a material capable of forming the wells 108 used in the present technology is preferable. Examples of such a material include an ultraviolet-curable resin, particularly a resin applicable to a 3D stereolithography method. Examples of a device used for the 3D stereolithography method include an ACCULAS (trademark) series stereolithography printer. The resin can be appropriately selected by a person skilled in the art. The resin can be obtained, for example, by curing a resin composition containing one or more selected from a silicone elastomer, an acrylic oligomer, an acrylic monomer, an epoxy-based oligomer, and an epoxy-based monomer with ultraviolet rays.

A material of another portion of the particle capture device 100 of the present technology can be appropriately selected by a person skilled in the art. For example, in a case where the particle 107 is a cell, the material is preferably a material that is not toxic to the cell.

Furthermore, in a case where fluorescence of the captured particles 107 is observed, it is preferable to use a material that does not emit autofluorescence beyond a permissible range.

Furthermore, it is preferable to use a material that makes observation of the particles 107 in the wells 108 possible. For observation of the particles 107, for example, at least a part of the particle capture device 100 can be constituted by a transparent material.

As a specific example of the material of the another portion of the particle capture device 100 of the present technology, for example, a material generally used in the technical field of the microchannel can be used. Examples of the material include glass such as borosilicate glass or quartz glass, a plastic resin such as an acrylic resin, a cycloolefin polymer, or polystyrene, and a rubber material such as PDMS. In a case where the particle capture device 100 of the present technology is constituted by a plurality of members, the plurality of members may be constituted by the same material or may be constituted by different materials.

In the present technology, the particle capture unit 103 can be replaceable. By making the particle capture unit 103 replaceable, a portion of the particle capture device 100 other than the particle capture unit 103 can be used repeatedly. The particle capture device 100 of the present technology can have a configuration in which the particle capture unit 103 inside the particle capture device 100 can be taken out. For example, by attaching a removable lid portion (not illustrated) to the particle capture device 100 and removing the lid portion, the particle capture unit 103 can be replaceable.

[Well 108]

In the particle capture device 100 according to the present technology, the wells 108 are portions where the particles 107 are captured. In the particle capture device 100 according to the present technology, the number of the wells 108 is not particularly limited and can be freely set according to a purpose. For example, a lower limit of the number of the wells 108 can be 2, particularly 10, more particularly 100, and still more particularly 1,000. An upper limit of the number of the wells 108 can be, for example, 1,000,000, particularly 800,000, more particularly 600,000, and still more particularly 500,000. A range of the number of the wells 108 may be determined by a value selected from either the above lower limit or the above upper limit, and can be for example, 1 to 1,000,000, particularly 10 to 800,000, more particularly 100 to 600,000, and still more particularly 1,000 to 500,000.

In the particle capture device 100 according to the present technology, the shape of the well 108 is not particularly limited, and a person skilled in the art can freely design the shape as long as the shape makes it possible to capture one particle 107. For example, the shape of an entrance of the particle 107 in the well 108 can be formed into a circle, an ellipse, or a polygon such as a triangle, a quadrangle (for example, a rectangle, a square, a parallelogram, or a rhombus), a pentagon, or a hexagon.

In the particle capture device 100 according to the present technology, arrangement of the wells 108 is not particularly limited, and can be freely designed according to a mode of the particle capture unit 103 and a purpose after particle capture. For example, the wells 108 can be arranged in a single row or in a plurality of rows at predetermined intervals, or can be arranged in a lattice form at predetermined intervals. The interval in this case can be appropriately selected by a person skilled in the art depending on, for example, the number of the particles 107 to be supplied and the number of the particles 107 to be captured. For example, the wells 108 can be designed such that the interval is 20 μm to 300 μm, preferably 30 μm to 250 μm, more preferably 40 μm to 200 μm, and still more preferably 50 μm to 150 μm.

The particles 107 captured in the wells 108 are subjected to observation, various reactions, various measurements, and the like depending on a purpose.

[Hole 114]

The holes 114 are preferably formed in the wells 108. The wells 108 and the second space 102 can be communicated with each other through the holes 114. Then, as described later, by discharging a fluid from the second discharge channel 106 connected to the second space 102, the particles 107 can be captured in the wells 108. The number of the holes 114 formed in each of the wells 108 can be, for example, 1 to 10, particularly 1 to 5, and more particularly 1 to 3. The number of the holes 114 formed in each of the wells 108 is preferably 1 or 2, and particularly 1 from a viewpoint of ease of manufacture.

Any shape can be adopted as the shape of the entrance of the hole 114. In the present technology, the entrance of the hole 114 means an opening of the hole 114 on a wall surface of the well 108 in which the hole 114 is formed. The shape of the entrance of the hole 114 can be formed into, for example, a circle, an ellipse, or a polygon such as a triangle, a quadrangle (for example, a rectangle, a square, a parallelogram, or a rhombus), a pentagon, or a hexagon. In the present technology, the shape of the entrance of the hole 114 is preferably a quadrangle, more preferably a rectangle or a square, and still more preferably a rectangle.

The entrance of the hole 114 is designed so as to have a size that prevents the particle 107 to be captured from passing through the hole 114 and advancing into the second space 102. For example, the minimum size of the entrance of the hole 114 can be less than the size of the particle 107.

For example, in a case where the shape of the entrance of the hole 114 is a rectangle, a size smaller than the size of the particle 107 to be captured (for example, the diameter of the particle 107) is used for a short side or a long side of the rectangle, particularly the short side of the rectangle. For example, the length of the short side of the rectangle can be designed to be 0.9 times or less, particularly 0.8 times or less, more particularly 0.7 times or less, and still more particularly 0.6 times or less the size of the particle 107 to be captured (for example, the diameter of the particle 107). The length of the short side of the rectangle also needs to be set so as not to interfere with a flow of a fluid, and can be for example, 0.01 times or more, particularly 0.1 times or more, and more particularly 0.3 times or more the size of the particle 107 to be captured.

For example, in a case where the shape of the entrance of the hole 114 is a circle, the hole 114 is formed so as to have a diameter smaller than the size of the particle 107 to be captured (for example, the diameter of the particle 107). For example, the diameter of the circle can be designed to be 0.8 times or less, particularly 0.7 times or less, and more particularly 0.6 times or less the size of the particle 107 to be captured (for example, the diameter of the particle 107). The diameter also needs to be set so as not to interfere with a flow of a fluid, and can be for example, 0.01 times or more, particularly 0.1 times or more, and more particularly 0.3 times or more the size of the particle 107 to be captured.

By designing the shape of the hole 114 as described above, it is possible to reliably capture the particles 107 while suppressing damage to the particles 107.

In the particle capture device 100 of the present technology, the shape of the entrance of the hole 114 is preferably a rectangle. The length of the long side of the rectangle can be designed to be preferably 1.2 times or more, more preferably 1.3 times or more, and still more preferably 1.5 times or more the length of the short side of the rectangle. Furthermore, the length of the long side of the rectangle can be designed to be, for example, preferably 5 times or less, more preferably 4 times or less, more preferably 3 times or less, and still more preferably 2.5 times or less the length of the short side of the rectangle. With such a slit shape, damage to the particles 107 when the particles 107 are captured in the wells 108 can be suppressed.

More specifically, for example, the shape of the entrance of the hole 114 can be designed to be a slit shape having a short side of 1 μm to 10 μm, particularly 2 μm to 8 μm, and a long side of 5 μm to 20 μm, particularly 6 μm to 18 μm.

The slit-shaped hole 114 as described above is particularly preferable in a case where the particle 107 is a cell. By the slit shape of the entrance of the hole 114, damage to a cell is suppressed while the cell is prevented from passing through the hole 114.

The hole 114 is preferably shallower from a viewpoint of workability. Meanwhile, the hole 114 is preferably deeper from a viewpoint of the strength of the particle capture unit 103. Therefore, in the particle capture device 100 according to the present technology, the depth of the hole 114 can be designed to be preferably 5 to 100 μm, more preferably 6 to 50 μm, and still more preferably 10 to 30 μm.

The hole 114 in the well 108 described above is not essential in the particle capture device 100 according to the present technology. For example, as in a second embodiment of the particle capture device 100 according to the present technology illustrated in FIG. 2, as a mode of the well 108, by forming an outlet of a fluid so as to be narrower than an inlet of the particle 107, the particle 107 can be captured without forming the hole 114.

(2) First space 101

At least the particle supply channel 104 and the first discharge channel 105 are connected to the first space 101. Furthermore, the first fluid supply channel 110 may be connected to the first space 101.

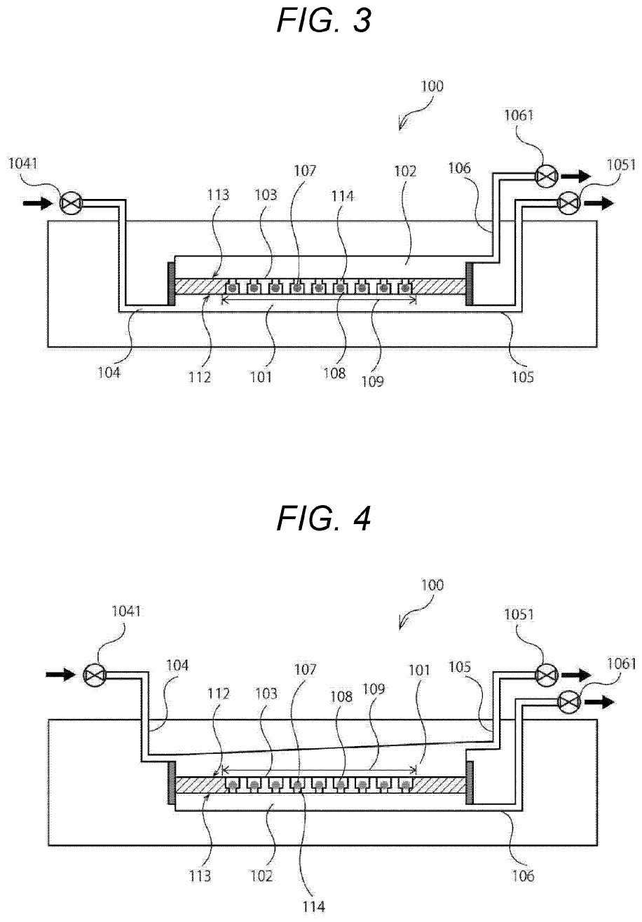

The first space 101 is formed above the second space 102 in the direction of gravity in the first embodiment illustrated in FIG. 1 and the second embodiment illustrated in FIG. 2, but is not limited thereto. For example, as in a third embodiment illustrated in FIG. 3, the first space 101 can be formed below the second space 102 in the direction of gravity.

By forming the first space 101 below the second space 102 in the direction of gravity, the particle 107 not captured in the well 108 settles in the direction of gravity action, that is, to a bottom of the first space 101. Therefore, It is possible to prevent the particle 107 not captured in the well 108 from staying around the well 108. As a result, further entrance of a particle 107 into the well 108 that has already captured another particle 107 can be suppressed.

Furthermore, a distance between a particle 107 captured in the well 108 and a particle 107 not captured in the well 108 is large. Therefore, when a particle 107 captured in the well 108 is subjected to observation, various reactions, various measurements, and the like, a particle 107 not captured in the well 108 can be easily removed. Furthermore, by focusing on a particle 107 captured in the well 108, the particle 107 captured in the well 108 can be subjected to observation, various reactions, various measurements, and the like without removing a particle 107 not captured in the well 108.

The first space 101 can be formed so as to have a larger cross-sectional area toward a downstream side as in a fourth embodiment of the particle capture device 100 according to the present technology illustrated in FIG. 4. By forming the particle capture device 100 in this way, a liquid containing the particles 107 is allowed to enter the first space 101 low and widely such that the particles 107 can be located at positions where the particles 107 can be easily captured in the wells 108, and the cross-sectional area gradually increases due to a sloping ceiling that becomes higher toward the first discharge channel 105. As a result, the flow velocity is reduced, and time for cells to settle can be gained. As a result, a probability that the particles 107 are captured by the particle capture unit 103 can be increased.

In this case, by forming an inlet from the particle supply channel 104 described later to the first space 101 to be low and wide (for example, height: 0.05 to 0.2 mm, width: 0.5 to 3 mm), and forming an outlet from the first space 101 to the first discharge channel 105 described later to be high and narrow (for example, height: 0.1 to 1 mm, width: 0.3 to 2 mm), the above effect can be obtained.

At this time, an opening cross-sectional area ratio between the inlet from the particle supply channel 104 described later to the first space 101 and the outlet from the first space 101 to the first discharge channel 105 described later is preferably inlet : outlet=1: 1.1 to 5.

[Particle supply channel 104]

A fluid containing the particles 107 is supplied from the particle supply channel 104. To the fluid supply channel, a valve 1041 and a container (not illustrated) that stores a fluid containing the particles 107 are connected. The particle supply channel 104 can be connected to a side surface of the first space 101. However, for example, although not illustrated, the particle supply channel 104 may be connected to an upper surface of the first space 101 or may be connected to a bottom surface of the first space 101 in a case where the first space 101 is below the second space in the direction of gravity as in the third embodiment illustrated in FIG. 3.

[First discharge channel 105]

The first discharge channel 105 can be connected to a valve 1051, the first discharge control unit 203 (not illustrated), and a pressure element such as a pump (not illustrated). The pump used in the present technology is preferably a pump capable of finely adjusting a suction force, and more preferably a pump capable of controlling a pressure around 1 kPa on the order of several tens of Pa. Such a pump is commercially available and examples thereof include KAL-200 (Hullstrap).

In the conventional single particle capture technique, a device having a structure in which the first discharge channel 105 is connected to the first space 101 may be used. However, the first discharge channel 105 of the conventional device is used for discharging particles 107 remaining in the first space 101 without being captured in the wells 108, or discharging particles 107 captured in the wells 108 after desired observation, various reactions, various measurements, and the like are performed after particle capture. That is, in the conventional single particle capture technique, by supplying a fluid containing the particles 107 from the particle supply channel 104 and discharging a fluid from the second discharge channel 106 while the valve 1051 of the first discharge channel 105 is closed, the particles 107 are captured in the wells 108.

Meanwhile, the present technology is characterized in that discharge of a fluid from the first discharge channel 105 is also performed simultaneously with supply of a fluid containing the particles 107 from the particle supply channel 104 and discharge of a fluid from the second discharge channel 106 while the valve 1051 of the first discharge channel 105 is opened. As a result, as described above, the particles 107 can be uniformly guided even to the wells 108 away from the particle supply channel 104 while the particles 107 to be captured are prevented from entering the holes 114 in the wells 108 or being damaged, and capture time of the particles 107 can be shortened.

The first discharge channel 105 is preferably disposed at a position facing the particle supply channel 104 with the particle capture region 109 interposed therebetween. By disposing the first discharge channel 105 in this way, the particles 107 can be uniformly guided even to the wells 108 away from the particle supply channel 104, and capture time of the particles 107 can be further shortened.

Note that in the particle capture device 100 according to the present technology, a fluid is discharged from the first discharge channel 105 during particle capture. However, as in the conventional single particle capture technique, the first discharge channel 105 in the particle capture device 100 according to the present technology can also be used for discharging particles 107 remaining in the first space 101 without being captured in the wells 108, or discharging particles 107 captured in the wells 108 after desired observation, various reactions, various measurements, and the like are performed after particle capture, of course.

In the particle capture device 100 according to the present technology, the flow velocity of a fluid flowing through the first discharge channel 105 during particle capture can be freely set as long as the effect of the present technology is not impaired. However, the flow velocity of a fluid flowing through the first discharge channel 105 is preferably set to be equal to or lower than the flow velocity of a fluid flowing through the second discharge channel 106 described later. By controlling the velocity of a fluid flowing through each of the first discharge channel 105 and the second discharge channel 106 in this way, the particles 107 can be uniformly guided even to the wells 108 away from the particle supply channel 104, and capture time of the particles 107 can be further shortened.

More specifically, during particle capture, the flow velocity of a fluid flowing through the first discharge channel 105: the flow velocity of a fluid flowing through the second discharge channel 106 is preferably 1:1 to 100, more preferably 1:2 to 50, and still more preferably 1:5 to 20.

A specific flow velocity of a fluid flowing through the first discharge channel 105 during particle capture can be appropriately set according to a suction pressure applied to the second discharge channel 106 described later, the flow velocity of a fluid flowing through the second discharge channel 106, the particle size of the particle 107 to be captured, the sizes and total number of the holes 114 of the wells 108, and the like.

Note that the flow velocity of a fluid flowing through the first discharge channel 105 can be controlled by controlling a suction pressure applied to the first discharge channel 105. For example, in a case where the particle capture device 100 according to the present technology includes the first discharge control unit 203, the first discharge control unit 203 can control the suction pressure applied to the first discharge channel 105.

The suction pressure applied to the first discharge channel 105 can also be appropriately set according to a suction pressure applied to the second discharge channel 106 described later, the flow velocity of a fluid flowing through the second discharge channel 106, the particle size of the particle 107 to be captured, the sizes and total number of the holes 114 of the wells 108, and the like.

In the particle capture device 100 according to the present technology, it is also possible to change the suction pressure applied to the first discharge channel 105 at a predetermined cycle during particle capture. By changing the suction pressure applied to the first discharge channel 105 at a predetermined cycle, the particles 107 can be prevented from staying in the first space 101, and capture time of the particles 107 into the wells 108 can be further shortened.

Examples of a method for changing the suction pressure applied to the first discharge channel 105 at a predetermined cycle include a method for superimposing pressure changes at a predetermined cycle while a constant suction pressure is applied to the first discharge channel 105.

[First fluid supply channel 110]

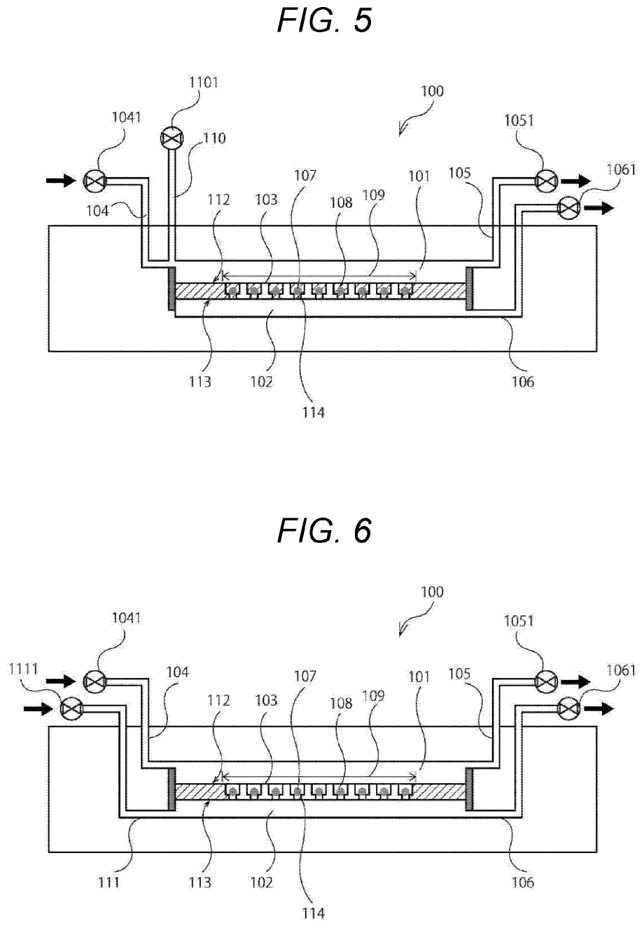

To the first space 101, in addition to the particle supply channel 104 and the first discharge channel 105, the first fluid supply channel 110 can also be connected as in a fifth embodiment of the particle capture device 100 according to the present technology illustrated in FIG. 5. From the first fluid supply channel 110, a fluid containing no particle 107 to be captured is supplied.

In the particle capture device 100 according to the present technology, by supplying a fluid containing no particle 107 to be captured from the first fluid supply channel 110 to the first space 101 during particle capture, it is possible to form a flow that flows while sandwiching a fluid containing a particle 107 to be captured supplied from the particle supply channel 104 between a laminar flow of the fluid containing no particle 107 to be captured and the particle capture unit 103 from the first fluid supply channel 110 toward the first discharge channel 105. As a result, the particles 107 can be uniformly guided even to the wells 108 away from the particle supply channel 104, and capture time of the particles 107 can be further shortened.

In this case, as the fluid supplied from the first fluid supply channel 110, a generally used buffer solution can be used. Examples of the buffer solution include PBS and HEPES. For example, in a case where cells are supplied from the particle supply channel 104 using a cell culture medium (for example, RPMI1640 or DMEM), the medium containing sugar has a higher specific gravity than the buffer solution. Therefore, a sample solution containing the cells can be conveyed so as to crawl on a surface of the wells 108.

Furthermore, in the particle capture device 100 according to the present technology, after the particles 107 are captured in the wells 108, by supplying a drug from the first fluid supply channel 110 or circulating a medium such as RPMI1640 or DMEM, it is also possible to stimulate the captured particles 107 with the drug or to culture the captured particles 107 for a long time, for example. At this time, another substance such as FBS may be added to the medium such as RPMI1640 or DMEM. In a case where FBS is added, the ratio of FBS may be, for example, 1% to 15%, and particularly 10%. In a case where the particles 107 are fluorescently stained and observed, for example, D-PBS (−), Live Cell Imaging Solution (ThermoFisher SCIENTIFIC), or FluoroBrite (trademark) DMEM (ThermoFisher SCIENTIFIC) having low autofluorescence may be used.

(3) Second space 102

At least the second discharge channel 106 is connected to the second space 102. Furthermore, to the second space 102, the second fluid supply channel 111 can also be connected.

[Second discharge channel 106]

The second discharge channel 106 can be connected to a valve 1061, the second discharge control unit 203 (not illustrated), and a pressure element such as a pump (not illustrated). The pump used in the present technology is preferably a pump capable of finely adjusting a suction force, and more preferably a pump capable of controlling a pressure around 1 kPa on the order of several tens of Pa. Such a pump is commercially available and examples thereof include KAL-200 (Hullstrap).

The flow velocity of a fluid flowing through the second discharge channel 106 during particle capture can also be appropriately set according to the particle size of the particle 107 to be captured, the sizes and total number of the holes 114 of the wells 108, and the like as long as the effect of the present technology is not impaired.

Note that the flow velocity of a fluid flowing through the second discharge channel 106 can be controlled by controlling a suction pressure applied to the second discharge channel 106. For example, in a case where the particle capture device 100 according to the present technology includes the second discharge control unit 203, the second discharge control unit 203 can control the suction pressure applied to the second discharge channel 106.

The suction pressure applied to the second discharge channel 106 can be freely set as long as the effect of the present technology is not impaired, but is preferably 0.001 to 1 kPa, more preferably 0.005 to 0.5 kPa, and for example, in a case where cells are to be captured, still more preferably 0.01 to 0.1 kPa.

In the particle capture device 100 according to the present technology, it is also possible to change the suction pressure applied to the second discharge channel 106 at a predetermined cycle during particle capture. By changing the suction pressure applied to the second discharge channel 106 at a predetermined cycle, the particles 107 can be prevented from staying in the first space 101, and capture time of the particles 107 into the wells 108 can be further shortened.

Examples of a method for changing the suction pressure applied to the second discharge channel 106 at a predetermined cycle include a method for superimposing pressure changes at a predetermined cycle while a constant suction pressure is applied to the second discharge channel 106.

[Second fluid supply channel 111]

To the second space 102, in addition to the second discharge channel 106, the second fluid supply channel 111 can also be connected as in a sixth embodiment of the particle capture device 100 according to the present technology illustrated in FIG. 6. From the second fluid supply channel 111, a fluid containing no particle 107 to be captured is supplied.

By discharging a fluid from the second fluid supply channel 111 when the particles 107 captured in the wells 108 are discharged from the first discharge channel 105 after desired observation, various reactions, various measurements, and the like are performed after particle capture, an extrusion pressure is applied to the particles 107 captured in the wells 108. Therefore, the particles 107 can be discharged from the wells 108 more smoothly. Note that the particles 107 captured in the wells 108 can be discharged more efficiently by closing a valve of the particle supply channel 104 and the valve 1061 of the second discharge channel 106.

<2. Particle capture method>

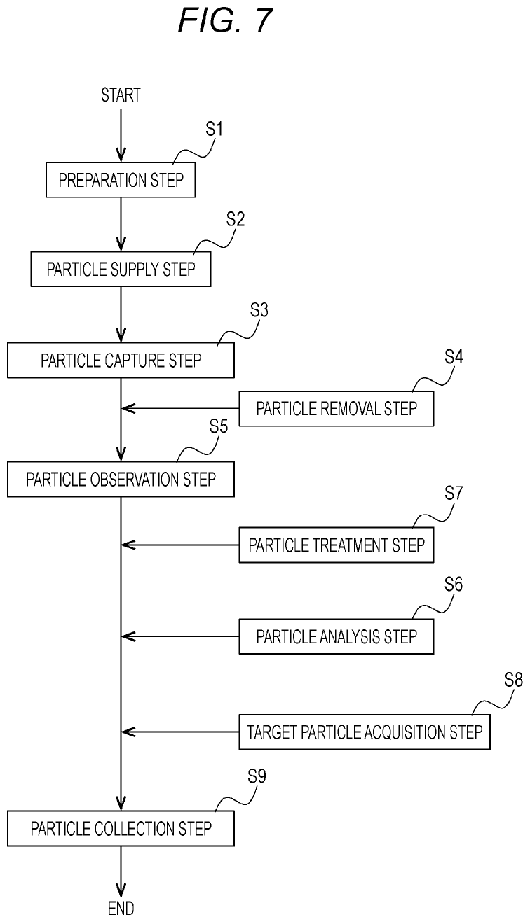

FIG. 7 is a flowchart of a particle capture method according to the present technology. The particle capture method according to the present technology is a method including at least a particle supply step S2 and a particle capture step S3. In addition, the particle capture method according to the present technology can also include a preparation step S1, a particle removal step S4, a particle observation step S5, a particle analysis step S6, a particle treatment step S7, a target particle acquisition step S8, a particle collection step S9, and the like. Hereinafter, each step will be described in detail in chronological order.

(1) Preparation step S1

The preparation step S1 is a step of preparing for particle capture. Specifically, a fluid such as a buffer solution containing no particle 107 to be captured is supplied to the first space 101 and the second space 102 of the particle capture device 100 described above, and the first space 101 and the second space 102 are filled with the fluid.

More specifically, a container containing a fluid to be filled in the first space 101 is connected to the particle supply channel 104 or the first fluid supply channel 110 connected to the first space 101, and the valve 1041 or 1101 of the particle supply channel 104 or the first fluid supply channel 110 is opened to fill the first space 101 with the fluid. Furthermore, a container containing a fluid to be filled in the second space 102 is connected to the second fluid supply channel 111 connected to the second space 102, and the valve 1111 of the second fluid supply channel 111 is opened to fill the second space 102 with the fluid.

(2) Particle supply step S2

The particle supply step S2 is a step of supplying a fluid containing the particles 107 to the first space 101. More specifically, a container containing a fluid containing the particles 107 is connected to the particle supply channel 104 connected to the first space 101, and the valve 1041 of the particle supply channel 104 is opened to supply the fluid containing the particles 107 to the first space 101.

(3) Particle capture step S3

The particle capture step S3 is a step of capturing the particles 107 in the wells 108. Specifically, the particles 107 are captured in the wells 108 by simultaneously performing discharge of a fluid from the first space 101 and discharge of a fluid from the second space 102. More specifically, while the particle supply step S2 is performed, the valve 1051 of the first discharge channel 105 connected to the first space 101 and the valve 1061 of the second discharge channel 106 connected to the second space 102 are opened to simultaneously perform discharge of a fluid from the first discharge channel 105 and discharge of a fluid from the second discharge channel 106.

At this time, by setting the discharge velocity of a fluid from the first space 101 to be equal to or lower than the discharge velocity of a fluid from the second space 102, the particles 107 can be uniformly guided even to the wells 108 away from the particle supply channel 104, and capture time of the particles 107 can be further shortened. The details of the discharge velocity of a fluid from each of the spaces are the same as the above-described flow velocity of a fluid flowing through each of the first discharge channel 105 and the second discharge channel 106 of the particle capture device 100 according to the present technology, and therefore description thereof is omitted here.

Furthermore, discharge of a fluid from the first space 101 and discharge of a fluid from the second space 102 can be performed by suction. The details of suction pressures for discharging a fluid from the first space 101 and discharging a fluid from the second space 102 are the same as the above-described suction pressures applied to the first discharge channel 105 and the second discharge channel 106 of the particle capture device 100 according to the present technology, and therefore description thereof is omitted here.

(4) Particle removal step S4

The particle removal step S4 is a step of removing the particles 107 not captured in the wells 108. Specifically, the particles 107 remaining in the first space 101 without being captured in the wells 108 are removed. The particle removal step is not an essential step in the particle capture method according to the present technology. For example, in a case where only observation of particles 107 captured in the wells 108 is performed using the particle capture device 100 according to the third embodiment illustrated in FIG. 3, by focusing only on the particles 107 captured in the wells 108, even if there are particles 107 remaining in the first space 101 without being captured, observation is possible. Therefore, a particle observation step S7 described later and the like can be performed without performing the particle removal step S4.

More specifically, in the particle removal step S4, by opening the valve 1051 of the first discharge channel 105 connected to the first space 101 while the valve 1061 of the second discharge channel 106 connected to the second space 102 is closed to discharge a fluid containing the particles 107 remaining in the first space 101 from the first discharge channel 105, the particles 107 not captured in the wells 108 can be removed.

At this time, by supplying a fluid not containing the particles 107 to be captured, such as a buffer solution, from the particle supply channel 104 or, for example, in a case where the particle capture device 100 according to the fifth embodiment illustrated in FIG. 5 is used, from the first fluid supply channel 110 to the first space 101, the non-captured particles 107 can be removed from the first space 101 more smoothly.

(5) Particle observation step S5

The particle observation step S7 is a step of observing the particles 107 captured in the wells 108. The particles 107 can be observed using, for example, a microscope such as an inverted microscope. In the particle observation step S7, imaging and the like using an imaging element may be performed, if necessary.

For example, in a case where the particles 107 are observed using the particle capture device 100 according to the third embodiment illustrated in FIG. 3, by maintaining suction from the second discharge channel 106 while the valve 1061 of the second discharge channel 106 connected to the second space 102 is opened, a state in which the particles 107 are captured in the wells 108 is maintained. The particles 107 can be observed while this state is maintained. Note that the suction from the second discharge channel 106 in the particle observation step S7 is preferably performed at a pressure smaller than the suction pressure in the particle capture step S3 in order to reduce a damage to the particles 107.

Note that in a case where the particle capture device 100 according to an embodiment other than the third embodiment illustrated in FIG. 3 is used, the particles 107 captured in the wells 108 maintain a state of being captured in the wells 108 due to gravity unless an external force is applied to the particles 107. Therefore, it is not necessary to maintain the suction from the second discharge channel 106 when the particles 107 are observed. Furthermore, also in the particle capture device 100 according to the third embodiment illustrated in FIG. 3, the suction from the second discharge channel 106 is not essential because a state in which the particles 107 are captured in the wells 108 can be maintained even if the suction from the second discharge channel 106 is not maintained depending on the shapes of the wells 108, the sizes and specific gravity of the particles 107, the specific gravity of a fluid, and the like.

(6) Particle analysis step S6

In the particle analysis step S6, the particles 107 held in the wells 108 are analyzed. For example, the structure, properties, and the like of the particles 107 can be analyzed on the basis of a result observed in the particle observation step S5. Furthermore, by observing the particles 107 that have been subjected to the particle treatment step S7 described later again in the particle observation step S5, various analyses can be performed on the basis of an interaction of the particles 107 with another substance, for example.

(7) Particle treatment step S7

The particle treatment step S7 is a step of performing a treatment such as adding a drug to the particles 107 captured in the wells 108 or causing another substance to react with the particles 107 captured in the wells 108. Specifically, by supplying a fluid containing a drug or another substance from the particle supply channel 104 or, for example, in a case where the particle capture device 100 according to the fifth embodiment illustrated in FIG. 5 is used, from the first fluid supply channel 110 to the first space 101, the particles 107 captured in the wells 108 can be treated.

At this time, the second space 102 may be filled with the buffer solution and the like used when the particles 107 are captured. However, in order to efficiently treat the particles 107, a fluid containing a drug or another substance may also be supplied to the second space 102. Specifically, for example, in a case where the particle capture device 100 according to the sixth embodiment illustrated in FIG. 6 is used, a fluid containing a drug or another substance can be supplied from the second fluid supply channel 111 to the second space 102. Note that for example, the drug or the like supplied to the second space 102 may be the same as the drug or the like supplied to the first space 101, or a different type of drug or the like may be supplied to the second space 102 depending on a purpose.

(8) Target particle acquisition step S8

The target particle acquisition step S8 is a step of acquiring only a target particle 107 among particles 107 captured in the wells 108. For example, a target particle 107 can be selected on the basis of results of the particle observation step S7 and the particle analysis step S8, and the target particle 107 can be acquired with, for example, a single particle acquisition device such as a micromanipulator.

Note that the target particle acquisition step S8 is not an essential step. For example, in a case where it is necessary only to observe and analyze the particles 107 and it is not necessary to select the particles 107, the process can proceed to the particle collection step S9 without performing this step.

(9) Particle collection step S9

The particle collection step S9 is a step of collecting the particles 107 captured in the wells 108. For example, after the particle observation step S7, the particle analysis step S6, the particle treatment step S7, the target particle acquisition step S8, and the like are performed, if necessary, the unnecessary particles 107 are collected. Specifically, the particles 107 are collected from the first discharge channel 105 connected to the first space 101.

More specifically, by performing suction from the first discharge channel 105 while the valve 1061 of the second discharge channel 106 connected to the second space 102 is closed, the particles 107 captured in the wells 108 can be collected from the first discharge channel 105.

At this time, for example, in a case where the particle capture device 100 according to the sixth embodiment illustrated in FIG. 6 is used, it is preferable to supply a fluid not containing the particles 107 to be captured, such as a buffer solution, from the second fluid supply channel 111 connected to the second space 102. By forming a flow of a fluid from the second fluid supply channel 111 toward the second space 102 through the second space 102, the particles 107 in the wells 108 can be pushed out, and the particles 107 can be collected from the first discharge channel 105 more smoothly.

<3. Microscope system 200>

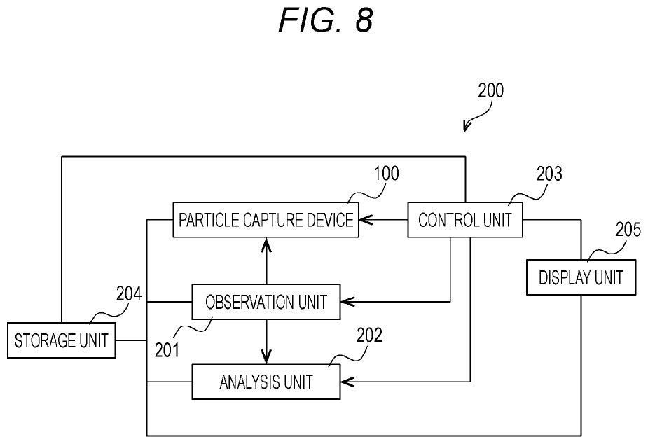

FIG. 8 is a block diagram of a microscope system 200 according to the present technology. The microscope system 200 according to the present technology includes at least the particle capture device 100 and an observation unit 201. Furthermore, the microscope system 200 can include an analysis unit 202, a control unit 203, a storage unit 204, a display unit 205, and the like, if necessary. Hereinafter, each unit will be described in detail. Note that the particle capture device 100 included in the microscope system 200 according to the present technology is the same as the above-described particle capture device 100 according to the present technology, and therefore description thereof is omitted here.

(1) Observation unit 201

The observation unit 201 observes the particles 107 captured in the wells 108. By observing the particles 107 captured in the wells 108, the shape, structure, color, and the like of the particles 107, the wavelength, intensity, and the like of light such as fluorescence generated from the particles 107 can be obtained. In the microscope system 200 according to the present technology, as the observation unit 201, a microscope or a photodetector can be used. As the microscope, an inverted microscope is preferably used. Furthermore, as the microscope, an optical microscope is preferably used. That is, in the microscope system 200 according to the present technology, as the observation unit 201, an inverted optical microscope is preferably used.

In the microscope system 200 according to the present technology, the observation unit 201 may include an imaging device. Examples of the imaging device include an imaging device including an image sensor, particularly a digital camera. Examples of the image sensor include a charge coupled device (CCD) and a complementary metal oxide semiconductor (CMOS).

Furthermore, the observation unit 201 may include various light sources, various lenses, various filters, various mirrors, and the like.

(2) Analysis unit 202

The microscope system 200 according to the present technology may further include the analysis unit 202, if necessary. The analysis unit 202 analyzes the particles 107 on the basis of information acquired by the observation unit 201. That is, a feature amount of the particles 107 can be calculated on the basis of the information acquired by the observation unit 201, and the mode, structure, property, and the like of the particles 107 can be analyzed on the basis of this feature amount.

Note that the analysis unit 202 is not essential for the microscope system 200 according to the present technology. The state and the like of the fine particles 107 can be analyzed using an external analysis device and the like on the basis of the information acquired by the observation unit 201. For example, the analysis unit 202 may be executed by a personal computer or a CPU, and can also be operated by the personal computer or the CPU by being stored as a program in a hardware resource including a recording medium (for example, non-volatile memory (USB memory), HDD, or CD). Furthermore, the analysis unit 202 may be connected to each unit of the microscope system 200 via a network.

(3) Control unit 203

The microscope system 200 according to the present technology may further include the control unit 203, if necessary. The control unit 203 can perform control of each unit included in the microscope system 200, such as control of supply of the particles 107 and a fluid to the particle capture device 100, control of discharge of the particles 107 and a fluid from the particle capture device 100, control of observation conditions in the observation unit 201, and control of observation conditions in the analysis unit 202.

For example, in the control of supply of the particles 107 and a fluid, by controlling valves of the particle supply channel 104, the first fluid supply channel 110, the second fluid supply channel 111, and the like of the particle capture device 100 and pressure elements connected to these channels, supply conditions of the particles 107 and a fluid can be controlled.

Furthermore, for example, in the control of discharge of the particles 107 and a fluid, by controlling valves of the first discharge channel 105, the second discharge channel 106, and the like of the particle capture device 100 and pressure elements connected to these channels, discharge conditions of the particles 107 and a fluid can be controlled. The particle capture device 100 according to the present technology captures the particles 107 in the wells 108 by discharging a fluid from the first discharge channel 105 and discharging a fluid from the second discharge channel 106. Therefore, by controlling discharge conditions of fluids from the first discharge channel 105 and the second discharge channel 106, as a result, capture conditions of the particles 107 can also be controlled.

Note that in the microscope system 200 according to the present technology, the control unit 203 is not essential, and each unit can be controlled by using an external control device. Furthermore, the control unit 203 may be connected to each unit of the microscope system 200 via a network.

(4) Storage unit 204

The microscope system 200 according to the present technology can include the storage unit 204 that stores various types of information. The storage unit 204 can store various pieces of data, conditions, and the like obtained in each unit of the microscope system 200, such as information data related to a capture state of the particles 107 in the particle capture device 100, observation data acquired by the observation unit 201, analysis data analyzed by the analysis unit 202, and control data in the control unit 203.

Note that in the microscope system 200 according to the present technology, the storage unit 204 is not essential, and an external storage device may be connected. As the storage unit 204, for example, a hard disk and the like can be used. Furthermore, the storage unit 204 may be connected to each unit of the microscope system 200 via a network.

(5) Display unit 205

The microscope system 200 according to the present technology may include the display unit 205 that displays various types of information. The display unit 205 can display various pieces of data, conditions, and the like obtained in each unit of the microscope system 200, such as information data related to a capture state of the particles 107 in the particle capture device 100, observation data acquired by the observation unit 201, analysis data analyzed by the analysis unit 202, and control data in the control unit 203.

In the microscope system 200 according to the present technology, the display unit 205 is not essential, and an external display device may be connected. As the display unit 205, for example, a display or a printer can be used. Furthermore, the display unit 205 may be connected to each unit of the microscope system 200 via a network.

Note that the present technology may have the following configurations.
[1]

A particle capture device including:

a particle capture unit having a particle capture region including a plurality of wells that captures particles, and dividing a space into a first space and a second space;

a particle supply channel which is connected to the first space and through which a fluid containing the particles is supplied;

a first discharge channel which is connected to the first space and through which a fluid is discharged from the first space; and

a second discharge channel which is connected to the second space and through which a fluid is discharged from the second space, in which

the particles are captured in the wells by simultaneous discharge of fluids from the first discharge channel and the second discharge channel.
[2]

The particle capture device according to [1], in which the first discharge channel is disposed at a position facing the particle supply channel with the particle capture region interposed therebetween.
[3]

The particle capture device according to [1] or [2], in which a flow velocity of a fluid flowing through the first discharge channel is equal to or less than a flow velocity of a fluid flowing through the second discharge channel during particle capture.
[4]

The particle capture device according to [3], in which the flow velocity of a fluid flowing through the first discharge channel: the flow velocity of a fluid flowing through the second discharge channel is 1:1 to 100 during particle capture.
[5]

The particle capture device according to any one of [1] to [4], in which a suction pressure applied to the first discharge channel and/or a suction pressure applied to the second discharge channel during particle capture is changed at a predetermined cycle.
[6]

The particle capture device according to any one of [1] to [5], in which the first space has a larger cross-sectional area toward a downstream side.
[7]

The particle capture device according to any one of [1] to [6], in which holes are formed in the wells, and the wells and the second space are communicated with each other through the holes.
[8]

The particle capture device according to any one of [1] to [7], in which the first space is formed above the second space in the direction of gravity during particle capture.
[9]

The particle capture device according to any one of [1] to [8], in which

a fluid is discharged by being sucked through the first discharge channel and the second discharge channel, and

the particles are captured in the wells by simultaneous discharge of fluids from the first discharge channel and the second discharge channel.
[10]

A particle capture method including:

a particle supply step of supplying a fluid containing particles to a first space; and

a particle capture step of capturing particles in a plurality of wells in a particle capture region formed in a particle capture unit dividing a space into the first space and a second space by simultaneously performing discharge of a fluid from the first space and discharge of a fluid from the second space.
[11]

The particle capture method according to [10], in which a discharge velocity of a fluid from the first space is equal to or less than a discharge velocity of a fluid from the second space.
[12]

The particle capture method according to [10] or [11], in which in the particle capture step, the discharge of a fluid from the first space and the discharge of a fluid from the second space are performed by suction.
[13]

A microscope system including:

a particle capture device including:

a particle capture unit having a particle capture region including a plurality of wells that captures particles, and dividing a space into a first space and a second space;

a particle supply channel which is connected to the first space and through which a fluid containing the particles is supplied;

a first discharge channel which is connected to the first space and through which a fluid is discharged from the first space; and

a second discharge channel which is connected to the second space and through which a fluid is discharged from the second space,

the particles being captured in the wells by simultaneous discharge from the first discharge channel and the second discharge channel; and

an observation unit that observes particles captured in the wells.
[14]

The microscope system according to [13], further including an analysis unit that analyzes the particles on the basis of information acquired from the observation unit.
[15]

The microscope system according to [13] or [14], further including:

a first discharge control unit that controls discharge via the first discharge channel during particle capture; and

a second discharge control unit that controls discharge via the second discharge channel during particle capture.

### REFERENCE SIGNS LIST

100 Particle capture device
101 First space
102 Second space
103 Particle capture unit
104 Particle supply channel
105 First discharge channel
106 Second discharge channel
108 Well
110 First fluid supply channel
111 Second fluid supply channel
114 Hole
S1 Preparation step
S2 Particle supply step
S3 Particle capture step
S4 Particle removal step
S5 Particle observation step
S6 Particle analysis step
S7 Particle treatment step
S8 Target particle acquisition step
S9 Particle collection step
200 Microscope system
201 Observation unit
202 Analysis unit
203 Control unit
204 Storage unit
205 Display unit

1. A particle capture device comprising:
a particle capture unit having a particle capture region including a plurality of wells that captures particles, and dividing a space into a first space and a second space;
a particle supply channel which is connected to the first space and through which a fluid containing the particles is supplied;
a first discharge channel which is connected to the first space and through which a fluid is discharged from the first space; and
a second discharge channel which is connected to the second space and through which a fluid is discharged from the second space, wherein
the particles are captured in the wells by simultaneous discharge of fluids from the first discharge channel and the second discharge channel.

2. The particle capture device according to claim 1, wherein the first discharge channel is disposed at a position facing the particle supply channel with the particle capture region interposed therebetween.

3. The particle capture device according to claim 1, wherein a flow velocity of a fluid flowing through the first discharge channel is equal to or less than a flow velocity of a fluid flowing through the second discharge channel during particle capture.

4. The particle capture device according to claim 3, wherein the flow velocity of a fluid flowing through the first discharge channel : the flow velocity of a fluid flowing through the second discharge channel is 1:1 to 100 during particle capture.

5. The particle capture device according to claim 1, wherein a suction pressure applied to the first discharge channel and/or a suction pressure applied to the second discharge channel during particle capture is changed at a predetermined cycle.

6. The particle capture device according to claim 1, wherein the first space has a larger cross-sectional area toward a downstream side.

7. The particle capture device according to claim 1, wherein holes are formed in the wells, and the wells and the second space are communicated with each other through the holes.

8. The particle capture device according to claim 1, wherein the first space is formed above the second space in a direction of gravity during particle capture.

9. The particle capture device according to any one of claim 1, wherein
a fluid is discharged by being sucked through the first discharge channel and the second discharge channel, and
the particles are captured in the wells by simultaneous discharge of fluids from the first discharge channel and the second discharge channel.

10. A particle capture method comprising:
a particle supply step of supplying a fluid containing particles to a first space; and
a particle capture step of capturing particles in a plurality of wells in a particle capture region formed in a particle capture unit dividing a space into the first space and a second space by simultaneously performing discharge of a fluid from the first space and discharge of a fluid from the second space.

11. The particle capture method according to claim 10, wherein a discharge velocity of a fluid from the first space is equal to or less than a discharge velocity of a fluid from the second space.

12. The particle capture method according to claim 10, wherein in the particle capture step, the discharge of a fluid from the first space and the discharge of a fluid from the second space are performed by suction.

13. A microscope system comprising:
a particle capture device including:
a particle capture unit having a particle capture region including a plurality of wells that captures particles, and dividing a space into a first space and a second space;
a particle supply channel which is connected to the first space and through which a fluid containing the particles is supplied;
a first discharge channel which is connected to the first space and through which a fluid is discharged from the first space; and
a second discharge channel which is connected to the second space and through which a fluid is discharged from the second space,
the particles being captured in the wells by simultaneous discharge from the first discharge channel and the second discharge channel; and
an observation unit that observes particles captured in the wells.

14. The microscope system according to claim 13, further comprising an analysis unit that analyzes the particles on a basis of information acquired from the observation unit.

15. The microscope system according to claim 13, further comprising:
a first discharge control unit that controls discharge via the first discharge channel during particle capture; and
a second discharge control unit that controls discharge via the second discharge channel during particle capture.
